(19) United States
(12) Patent Application Publication
(10) Pub. No.: US 2010/0028350 A1
(43) Pub. Date: Feb. 4, 2010

(54) PREVENTING IL-2 MEDIATED INFLAMMATION IN EPITHELIAL CELLS

(75) Inventors: Anthony M. Jevnikar, Ontario (CA); Caigan Du, Ontario (CA)

Correspondence Address: KNOBBE MARTENS OLSON & BEAR LLP, 2040 MAIN STREET, FOURTEENTH FLOOR, IRVINE, CA, 92614, US

(73) Assignee: London Health Sciences Centre Research Inc., London, ON (CA)

(21) Appl. No.: 12/281,474

(22) PCT Filed: Mar. 5, 2007
(86) PCT No.: PCT/CA2007/000339

§ 371 Date: Jun. 1, 2009

Related U.S. Application Data
(60) Provisional application No. 60/778,409, filed on Mar. 3, 2006.

(51) Int. Cl. A61K 39/395; A61K 31/713; A61P 37/00; A61P 29/00; A61P 37/06; A61P 1/00

(52) U.S. Cl. 424/139.1; 514/44 A

(57) ABSTRACT

The present invention provides new methods for treating, minimizing and/or preventing inflammatory injury involving IL-2 activity on epithelial cells expressing IL-2 receptors. Methods for reducing or preventing inflammatory injury in a tissue/organ comprise inhibiting IL-2 receptor activity in cells of the tissue/organ.

### FIELD OF THE INVENTION

The invention relates to methods of inhibiting IL-2 mediated tissue/organ damage. More specifically, the invention relates to methods of preventing IL-2 mediated epithelial cell damage and cell death. The methods of the invention are useful in the treatment of inflammatory responses involving IL-2 receptor expressing epithelial cells and other non-T cells where such inflammation leads to cell damage and cell death.

### BACKGROUND OF THE INVENTION

Interleukin 2 is a well characterized cytokine with central roles in inflammation and immune signalling. Interleukin 2 is expressed in inflammation. Its actions are generally mediated on T cells, B cells and natural killer cells which possess high affinity CD25 receptors, typically resulting in escalating functional activity of these target cells. Inflammation is a localized protective response elicited by tissues in response to injury, infection, or tissue destruction resulting in the destruction of the infectious or injurious agent and isolation of the injured tissue. A typical inflammatory response proceeds as follows: recognition of an antigen as foreign or recognition of tissue damage; synthesis and release of soluble inflammatory mediators; recruitment of inflammatory cells to the site of infection or tissue damage; destruction and removal of the invading organism or damaged tissue; and deactivation of the system once the invading organism or damage has been resolved.

Renal proximal tubular epithelial cells (PTEC) from mouse and human have been shown to express IL2 receptor α, β and γ chains on their surface. In addition, previous work has demonstrated IL2 receptor up-regulation with co-stimulation with interferon γ in human PTEC. Previous work using an immortalized mouse PTEC cell line (CS3.7) demonstrated direct tubular cell toxicity with exogenous IL2 administration (Du et al., Kidney International, Vol. 67 (2005), pp. 1-13). This appeared to be a direct result of down-regulation of c-FLIP expression, an endogenous inhibitor of the caspase cascade. With down-regulation of c-FLIP, caspase 8 activity was shown to increase, with concomitant increase in cellular apoptosis including Fas mediated fratricide.

Although the IL-2 receptor is expressed on human PTEC, it is currently not known whether human cells respond in a similar fashion since the previous work involved the use of an immortalized mouse cell line which may have different physiological characteristics and responses compared to human cells.

The down-regulation of T lymphocytes to prevent unwanted immune responses is known. For example U.S. Pat. No. 6,113,900 discloses methods of inhibiting allograft rejection where the inhibition is done to inhibit the proliferation of lymphocytes thus mitigating unwanted immune responses.

It is desirable to provide a method whereby undesired inflammatory responses are inhibited, minimized or averted in tissues/organs through the down-regulation of IL-2 effects on non-T cells, such as epithelial cells, that express IL-2 receptors.

### SUMMARY OF THE INVENTION

The present invention provides new methods for treating, minimizing and/or preventing inflammatory injury mediated via IL-2 activity on cells expressing IL-2 receptors. In aspects of the invention the cells are non-T cells. In further aspects of the invention the cells are epithelial cells.

According to an aspect of the present invention is a method for inhibiting IL-2 production in a patient in need thereof, wherein said method comprises administering an effective amount of an IL-2 blocker or IL-2 receptor blocker to said patient.

According to an aspect of the present invention is a method of treating an inflammatory disorder comprising the step of administering to a mammal a therapeutically effective amount of a composition comprising at least one agent that inhibit IL-2 receptor activation.

According to an aspect of the invention is a method for treating a subject having an inflammatory disease or condition comprising administering to the subject an IL-2 receptor antagonist in a daily amount of about 0.1 μmol to about 100 μmol per kg body weight, for at least a time effective to treat the subject for the inflammatory disease or condition.

According to an aspect of the present invention is a method for reducing or preventing inflammatory injury in a tissue/organ, the method comprising:

inhibiting IL-2 receptor activity in cells comprising said tissue/organ.

According to another aspect of the present invention is a method for increasing the viability of a tissue/organ allograft in a mammal, said method comprising:

blocking IL-2 receptor activity of epithelial cells of said tissue/organ allograft.

According to another aspect of the present invention is a method for reducing or preventing apoptosis of epithelial cells, said method comprising:

blocking IL-2 receptor activity in said cells.

According to yet a further aspect of the present invention is a method for reducing or preventing inflammation and related tissue/organ damage in a mammal, said method comprising:

administering an agent capable of binding to an IL-2 receptor on an epithelial cell such that IL-2 receptor signalling is reduced or prevented.

In aspects of the invention, the agent is an antibody that binds to the IL-2 receptor such that receptor signalling is prevented.

According to yet another aspect of the invention is a method to prevent kidney allograft rejection in a mammal receiving such allograft:

said method comprising administering to said mammal an effective amount of an agent that blocks IL-2 receptor activity in epithelial cells in said kidney.

According to still another aspect of the invention is a method of inhibiting rejection of an allograft in a mammal, comprising administering to said mammal an IL-2 receptor specific antibody in an amount effective to inhibit IL-2 receptor activity on epithelial cells of said allograft.

According to yet a further aspect of the present invention is a method for the treatment of inflammatory conditions in a subject, the method comprising administering to said subject a therapeutically effective amount of an agent that reduces/inhibits IL-2 receptor activity on non-T cells in said subject.

Another aspect of the invention is to provide methods of administering siRNA to a patient in need thereof, wherein the siRNA molecule is delivered in the form of a naked oligonucleotide, sense molecule, antisense molecule, or a vector, wherein the siRNA interacts with the IL-2 receptor gene or its transcripts on non-T cells, wherein the vector is a plasmid, cosmid, bacteriophage, or a virus, wherein the virus is for example, a retrovirus, an adenovirus, or other suitable viral vector.

Another aspect of the invention is to provide methods of administering miRNA to a patient in need thereof, wherein the miRNA molecule is delivered in the form of a naked oligonucleotide, sense molecule, antisense molecule, or a vector, wherein the miRNA interacts with the IL-2 receptor gene or its transcripts on non-T cells, wherein the vector is a plasmid, cosmid, bacteriophage, or a virus, wherein the virus is for example, a retrovirus, an adenovirus, or other suitable viral vector.

According to yet another aspect of the present invention is a method for screening to identify compounds that block IL-2 mediated IL-2 receptor activation, the method comprising:

providing a test compound to a human non-T cell culture; and

determining IL-2 receptor activation.

In aspects of the invention, the cell culture is an epithelial cell culture. In other aspects, the cell culture is a renal cell culture such as for example but not limited to renal proximal tubular epithelial cells (PTEC).

Still in another aspect, the invention provides methods of screening a test molecule for IL-2 receptor antagonist activity comprising, in any practical order, the steps of: determining the mRNA expression level of IL-2 receptor in a biological sample containing epithelial cells, thereby generating data for a pre-test level expression of IL-2 receptor mRNA; contacting the biological sample with the test molecule; determining the expression level of IL-2 receptor mRNA in a cell by determining the overall mRNA expression divided by the number of cells present in the sample, thereby generating data for a test level; and comparing the test level to the pre-test level expression of IL-2 receptor mRNA, wherein a decrease in expression of IL-2 receptor mRNA in the test level indicates IL-2 antagonist activity of the test molecule, wherein the expression level of IL-2 receptor can be determined by, for example, reverse transcription and polymerase chain reaction (RT-PCR), Northern hybridization, or microarray analysis.

Other features and advantages of the present invention will become apparent from the following detailed description and drawings. It should be understood, however, that the detailed description and drawing while indicating embodiments of the invention are given by way of illustration only, since various changes and modifications within the spirit and scope of the invention will become apparent to those skilled in the art from said detailed description.

### BRIEF DESCRIPTION OF THE DRAWINGS

The present invention will become more fully understood from the detailed description given herein and from the accompanying drawings, which are given by way of illustration only and do not limit the intended scope of the invention.

FIG. 1 shows an interleukin 2 dose response Annexin V—FITC FACS assay of human PTEC cell line HK-2. A. media control; B. 10 mg/mL IL-2; C. 20 ng/mL IL-2; D. 40 ng/mL IL-2.

FIG. 2 shows an interleukin 2 blocking study of HK-2 human PTEC. A. media control; B. TNFα+IFNγ+IL-2 positive control; C. IL-2 20 ng/mL treatment×24 hours; D. basiliximab 10 μg/mL pre-treatment×1 hr. followed by 24 hr treatment with media; E. basiliximab 10 μg/mL pre-treatment×1 hr followed by treatment with 20 ng/mL IL-2×24 hrs.

FIG. 3 shows a blocking study in mouse NG cell line assayed by Annexin V—FITC/7 AAD FACS. A. NG cells treated with 20 ng/mL mouse IL-2; B. mouse anti-CD25 MAb (10 μg/mL) pre-treatment×1 hr followed by 24 hr incubation with media; C. NG cells pretreated 1 hr with anti-CD25 followed by treatment with mouse IL-2 (20 ng/mL)×24 hrs.

FIG. 4 shows a demonstration of basiliximab effect in mouse cell. Annexin V-FITC/7 AAD FACS blocking assay similar to FIG. 3 with substitution of basiliximab pre-treatment with mouse IL-2 treatment×24 hrs.

### DETAILED DESCRIPTION OF THE PREFERRED EMBODIMENTS

The present invention is a novel method for the prevention of inflammatory mediated injury in tissues/organs comprising cells expressing the IL-2 receptor. Such method involves in general the inhibition of IL-2 receptor activity on non-T cells, more specifically, the method involves the inhibition of IL-2 binding with the IL-2 receptor on non-T cells, such as epithelial cells. Such method will lead to improved tissue and organ viability in inflammatory conditions. The method has use in the treatment of a variety of inflammatory conditions of tissues and organs. The method has use in the treatment of a variety of autoimmune disorders, as these involve inflammatory responses. For example, such disorders may include but not be limited to lupus, nephritis and inflammatory bowel disease. In other such non-limiting examples, the method has use for the increased success of organ transplantation.

Interleukin-2 (IL-2) is the main growth factor of T lymphocytes (THEZE et al. 1996, Immunol. Today 17:481-486). Human IL-2 is a protein of 133 amino acids (aa) composed of four α-helices connected by loops of various length; its tri-dimensional structure has been established. IL-2R is composed of three chains α, β and γ. IL-2Rα controls the affinity of the receptor. IL-2Rα and IL-2Rγ are responsible for IL-2 signal transduction. The different molecular areas of IL-2 interacting with the three chains of the IL-2 R have been defined. More specifically it has been determined that αhelix A as well as the NH₂ terminal area of IL-2 (residues 1 to 30) control the interactions IL-2/IL-2Rβ. (ECKENBERG et al. 1997, Cytokine 9:488-98): IL-2Rβ chain is most important in IL-2 signaling (THEZE et al. 1990). The effects of human interleukin-2 (IL-2) on its target cells are mediated through specific cell surface receptors (IL-2R) (TANIGUCHI et al. (1983) Nature 302:305-310; ROBB et al. (1984) Proc. Natl. Acad. Sci. USA 81:6486-6490; SMITH K A. 1988a. Interleukin-2; SMITH K A (1988b) Science 240:1169-1176). The IL-2R is heterotrimeric protein expressed on the surface of certain immune cells, such as lymphocytes, that binds and responds to interleukin 2. Three protein chains (α, β and γ) are non-covelently associated to form the IL-2R. The α and β chains are involved in binding IL-2, while signal transduction following cytokine interaction is carried out by the γ-chain, along with the β subunit. The β and γ chains of the IL-2R are members of the type I cytokine receptor family.

It is presently demonstrated that the exposure of human tubular epithelial cells of the kidney to exogenous IL-2 resulted in apoptosis related to a reduction in expression of the endogenous caspase-8 c-FLIP, with subsequent increased caspase-8 activation and fratricide. Furthermore, antibody directed at the high affinity IL-2 receptor (CD25) blocked IL-2 effects on TEC and attenuates cell death. This provides a new therapeutic application for IL-2R blocking antibody in having a protective effect on parenchymal cells of the graft rather than attenuating immune responses of T-cells in transplants, which is the conventional use for these antibodies. This has a role in epithelial cell protection in other forms of inflammation in which IL-2 may have a harmful effect on epithelial cells. This is the first report of direct IL-2 mediated cytotoxicity of human PTEC.

Using basiliximab, a commercially available IL-2 receptor antagonist, IL-2 mediated apoptosis was attenuated in human PTEC. The mechanism of apoptosis induction in human PTEC follows IL-2 exposure IL-2 treatment of PTEC results in down-regulation of c-FLIP and subsequent increase in caspase-8 activation through Fas activation.

The present invention demonstrates a previously unknown clinical utility for IL-2 receptor blockade in renal transplantation to prevent renal tubular injury from diverse forms of inflammation that may occur before, during, and after transplantation, including ischemia/reperfusion, cytokines, rejection and other agents. As well, chronic injury in allograft (CAN) may be improved with IL-2R blockade if low level exposure of TEC to cytokines contributes to longer term injury.

It is understood by one of skill in the art that the methods of the invention extend to any cells in tissues and organs that express the IL-2 receptor. Such tissues and organs may include but not be limited to heart, kidneys, liver, lungs, pancreas, small intestine, skin, bone, veins and tendons. In aspects of the invention, the organ is kidney.

As the invention has demonstrated the successful blockage of epithelial IL-2 receptors, in one aspect using antibodies, it is expected by one of skill in the art that this is a broad treatment for any inflammatory condition or autoimmune disease to target IL-2 expressing epithelial cells to reduce/prevent/treat inflammation. Thus such inhibition is used to improve tissue and organ viability in inflammatory conditions and autoimmune disorders involving inflammatory responses. As such, the present invention in the blockade of IL-2 receptors on epithelial cells (non T cells) can also be used successfully in organ transplantation where improved media for preserving, reperfusing and storing organs prior to transplant are required. Such media that comprises an IL-2 receptor antagonist may provide organs with increased functionality upon transplant and after transplant. When organs are harvested for transplantation, the ensuing period of hypoxia, followed by reperfusion of the organ, is accompanied by substantial tissue damage, including endothelial cell apoptosis and parenchymal dysfunction. Such ischemia/reperfusion (I/R) injury may involve inflammatory reactions, activation of apoptotic pathways, activation of the complement system, and activation of immuno-inflammatory genes. The compositions and methods of the present invention have utility for any desired tissue/organ transplantation to attenuate, prevent inflammation in order to provide a more successful transplant outcome.

### Antacionist Therapy

In one embodiment, the method of the present invention involves the use of an antagonist to the IL-2 receptor. It is understood to those of skill in the art that any antagonist compound/molecule/protein that binds to the IL-2 receptor such that IL-2 binding is prevented and also does not cause IL-2 receptor activation leading to intracellular signalling is encompassed in the methods of the present invention.

In particular, an antagonist of an IL-2 Receptor (IL-2R) is an agent that specifically binds to the IL-2R, or a component thereof, and inhibits a biological function of the IL-2 receptor or the component. Exemplary functions that can be inhibited are the binding of IL-2 to the IL-2R, the intracellular transmission of a signal from binding of IL-2, and proliferation and/or activation of lymphocytes such as T cells in response to IL-2. In one embodiment, IL-2R antagonists of use in the methods disclosed herein inhibit at least one of these functions. Alternatively, IL-2R antagonist of use in the methods disclosed herein can inhibit more than one or all of these functions.

Any compound that functions as an IL-2 receptor antagonist and is suitable for administration in accordance with the methods of the present invention may be employed in the present invention. Antagonists need not completely abolish IL-2-induced biological activity to be useful. Rather, a given antagonist may reduce a biological activity of IL-2. Derivatives, mutants/muteins, and other variants of IL-2 that function as IL-2 antagonists may be employed. Peptides (which may or may not be muteins) derived from IL-2 that bind to an IL-2R without inducing transduction of a biological signal find use herein. Such peptides function as inert blockers, interfering with the binding of biologically active endogenous IL-2 to cell surface receptors. IL-2-induced signal transduction thereby is inhibited. Muteins or other antagonists that induce a biological response at a reduced level or to a lesser degree, compared to the response induced by native IL-2, also find use as IL-2 antagonists. The structure, nucleic acid and amino acid sequence of human IL-2 is found for example in U.S. Pat. No. 6,825,334 (the disclosure of which is incorporated herein by reference in its entirety). The preparation of IL-2 antagonists can be as that described for IL-4, including IL-2 muteins, as described in Muller et al., J. Mol. Biol., 237:423-436, 1994; U.S. Pat. No. 6,028,176, and U.S. Pat. No. 5,723,118, which are each incorporated by reference herein. Any suitable assay, including in vitro assays, can be utilized to determine whether a given compound inhibits an IL-2-induced biological activity. An alternative involves use of conventional binding assay techniques to test an antagonist for the ability to inhibit binding of IL-2 to cells expressing native or recombinant IL-2 receptors. The ability of an IL-2 antagonist to inhibit IL-2-induced damage to epithelium, such as lung epithelium or intestinal epithelium (which may result in loss of barrier function), may be confirmed in any of a number of suitable assays.

Thus to reduce or inhibit the IL-2 induced activity, an appropriate IL-2 peptide inhibitor of IL-2 may be introduced to block the interaction of the IL-2 with the IL-2R.

In one aspect, as the IL-2 receptor-specific antagonist, are antibodies (in aspects monoclonal) which are specific in vitro and in vivo for the IL-2 receptor on cells such as epithelial cells. Antibodies specific for the IL-2 receptor on T-lymphocytes can be made using standard techniques, or can be purchased, e.g., from Novartis (Simulect) and Roche (Zenapax)). In one example, an IL-2 receptor antagonist is an antibody that specifically binds Tac (p55), such as Zenapax™. Other anti-p55 agents include the chimeric antibody Basiliximab (Simulect™), BT563 (see Baan et al., Transplant. Proc. 33:224-2246, 2001), and 7G8. An exemplary human anti-p55 antibody of use in the methods of the invention is HuMax-TAC, by Genmab. In another example, an IL-2 receptor antagonist is an antibody that specifically binds the p75 or βsubunit of the IL-2R. Additional antibodies that specifically bind the IL-2 receptor are known in the art, for example, see U.S. Pat. No. 5,011,684; U.S. Pat. No.5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559. The antibody can be monoclonal or polyclonal, and can be derived from any suitable animals.

Production and initial screening of monoclonal antibodies to yield those specific for the IL-2 receptor can be carried out as described in Uchiyama et al. (1981) J. Immunol. 126 (4), 1393; this method, briefly, is as follows. Human cultured T-lymphocytes are injected into mammals, e.g., mice, and the spleens of the immunized animals are removed and the spleen cells separated and then fused with immortal cells, e.g., mouse or human myeloma cells, to form hybridomas.

The antibody-containing supernatants from the cultured supernatants are then screened for those specific for the IL-2 receptor, using a complement-dependent cytotoxicity test, as follows. Human T-lymphocytes and EBV transformed B-lymphocytes are labelled with ⁵¹Cr sodium chromate and used as target cells; these cells are incubated with hybridoma culture supernatants and with complement, and then the supernatants are collected and counted with a gamma counter. Those supernatants exhibiting toxicity against activated T-lymphocytes, but not resting T- or B-lymphocytes, are selected, and then subjected to a further screening step to select those supernatants containing antibody which precipitates (i.e., is specifically reactive with) the 50 kilodalton glycoprotein IL-2 receptor (described in detail in Leonard et al. (1983) P.N.A.S. USA 80, 6957). The desired anti-IL-2 receptor antibody is purified from the supernatants using conventional methods.

In another example, an IL-2 receptor antagonist is a peptide antagonist that is not an antibody. Peptide antagonists of the IL-2 receptor, including antagonists of Tac (p55) and p75 (IL-2Rβ) are also known. For example, peptide antagonists for p55 and p75 are disclosed in U.S. Pat. No. 5,635,597. These peptides are also of use in the methods disclosed herein. In a further example, an IL-2 receptor antagonist is a chemical compound or small molecule that specifically binds to the IL-2 receptor and inhibits a biological function of the receptor.

To be more specific with the embodiment of an antibody IL-2 receptor antagonist, the general methodology for making monoclonal antibodies by hybridomas is well known. Thus one of skill in the art may purchase or make a suitable antibody in accordance with the methods and compositions of the invention. Immortal, antibody-producing cell lines can also be created by techniques other than fusion, such as direct transformation of B lymphocytes with oncogenic DNA; or transfection with Epstein-Barr virus. See, e.g., M. SCHREEER et al., “Hybridoma Techniques” (1980); HAMMERLING et al., “Monoclonal Antibodies And T-cell Hybridomas” (1981); KENNETT et al., “Monoclonal Antibodies” (1980); see also U.S. Pat. Nos. 4,341,761; 4,399,121; 4,427,783; 4,444,887; 4,451,570; 4,466,917; 4,472,500; 4,491,632; 4,493,890. Panels of monoclonal antibodies produced against IL-2 peptides can be screened for various properties; i.e., isotype, epitope, affinity, etc. Of particular interest are monoclonal antibodies that modulate the activity of IL-2 or peptides thereof. Such monoclonals can be readily identified in cellular proliferation assays. High affinity antibodies are also useful when immunoaffinity purification of native or recombinant IL-2 or IL-2 peptides is possible. The anti-IL-2 receptor antibody used in the therapeutic methods of this invention is an affinity purified polyclonal antibody and in other aspects, the antibody is a monoclonal antibody (mAb). Methods for producing polyclonal anti-polypeptide antibodies are well-known in the art. See U.S. Pat. No. 4,493,795 to Nestor et al. A monoclonal antibody, typically containing Fab and/or F(ab′)₂ portions of useful antibody molecules, can be prepared using the hybridoma technology described in Antibodies—A Laboratory Manual, Harlow and Lane, eds., Cold Spring Harbor Laboratory, New York (1988), which is incorporated herein by reference. Briefly, to form the hybridoma from which the monoclonal antibody composition is produced, a myeloma or other self-perpetuating cell line is fused with lymphocytes obtained from the spleen of a mammal hyperimmunized with an IL-2 peptide or IL-2 R-binding portion thereof. Splenocytes are typically fused with myeloma cells using polyethylene glycol (PEG) 6000. Fused hybrids are selected by their sensitivity to HAT. Hybridomas producing a monoclonal antibody useful in practicing this invention are identified by their ability to immunoreact with the present IL-2 mutant or peptide and their ability to inhibit specified IL-2 activity in target cells.

A monoclonal antibody useful in practicing the present invention can be produced by initiating a monoclonal hybridoma culture comprising a nutrient medium containing a hybridoma that secretes antibody molecules of the appropriate antigen specificity. The culture is maintained under conditions and for a time period sufficient for the hybridoma to secrete the antibody molecules into the medium. The antibody-containing medium is then collected. The antibody molecules can then be further isolated by well-known techniques. Media useful for the preparation of these compositions are both well-known in the art and commercially available and include synthetic culture media, inbred mice and the like. An exemplary synthetic medium is Dulbecco's minimal essential medium (DMEM; DULBECCO et al., Virol. 8:396 (1959)) supplemented with 4.5 gm/1 glucose, 20 mm glutamine, and 20% fetal calf serum. An exemplary inbred mouse strain is the Balb/c. Methods for producing monoclonal anti-IL-2 antibodies are also well-known in the art. See NIMAN et al., Proc. Natl. Acad. Sci. USA, 80:4949-4953 (1983). Typically, the present IL-2 peptide or a peptide analog is used either alone or conjugated to an immunogenic carrier, as the immunogen in the before described procedure for producing anti-IL-2 monoclonal antibodies. The hybridomas are screened for the ability to produce an antibody that immuoreacts with the IL-2 mutant or peptide analog.

As discussed above, the IL-2 peptides or their binding partners or other ligands or agents exhibiting either mimicry or antagonism to IL-2 receptor or control over its production, may be prepared in pharmaceutical compositions, with a suitable carrier and at a strength effective for administration by various means to a patient experiencing an adverse medical condition associated with undesirable levels of IL-2 for the treatment thereof. A variety of administrative techniques may be utilized, among them parenteral techniques such as subcutaneous, intravenous and intraperitoneal injections, catheterizations and the like. Average quantities of the IL-2 peptides or their subunits may vary and in particular should be based upon the recommendations and prescription of a physician or veterinarian in the case of an animal.

The present invention further contemplates the use of therapeutic compositions which are useful in practicing the therapeutic methods of this invention. In one embodiment, the therapeutic composition includes, in admixture, a pharmaceutically acceptable excipient (carrier) and one or more of a IL-2 peptide, a purified peptide or a derivative thereof or a polypeptide analog thereof or fragment thereof, as described herein as an active ingredient to antagonize the IL-2 receptor. In a preferred embodiment, the therapeutic composition comprises an active compound containing a purified peptide capable of modulating the specific binding of the present IL-2 with the IL-2R. The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

The use of the compositions may be by administration in a manner compatible with the dosage formulation, and in a therapeutically effective amount. The quantity to be administered depends on the subject to be treated, capacity of the subject's immune system to utilize the active ingredient, and degree of modulation of IL-2 binding capacity desired. Precise amounts of active ingredient required to be administered depend on the judgment of the practitioner and are peculiar to each individual. However, suitable dosages may range from about 0.1 to 20, preferably about 0.5 to about 10, and more preferably one to several, milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated. The use of the therapeutic compositions may be by administration in a composition which further includes an effective amount of the IL-2 antagonist or analog thereof, and one or more of other active ingredients

The pharmaceutical compositions comprising one or more IL-2 receptor antagonists for administration to subjects in a biologically compatible form suitable for administration in vivo. By “biologically compatible form suitable for administration in vivo” is meant a form of the substance to be administered in which any toxic effects are outweighed by the therapeutic effects. Administration of a therapeutically active amount of the pharmaceutical compositions of the present invention, or an “effective amount”, is defined as an amount effective at dosages and for periods of time, necessary to achieve the desired result of preventing or minimizing the inflammatory response. In aspects this is a reduction or prevention of cell death in tissues and organs where the cells express IL-2 receptors. A therapeutically effective amount of a substance may vary according to factors such as the disease state/health, age, sex, and weight of the recipient, and the inherent ability of the particular antagonist to elicit a desired response. As described herein, dosage regima may be adjusted to provide the optimum therapeutic response. For example, several divided doses may be administered daily or on at periodic intervals, and/or the dose may be proportionally reduced as indicated by the exigencies of the therapeutic situation. The amount of IL-2 receptor antagonist for administration will depend on the route of administration, time of administration and varied in accordance with individual subject responses. Suitable administration routes are intramuscular injections, subcutaneous injections, intravenous injections or intraperitoneal injections, oral and intranasal administration.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the disclosure of which is incorporated herein by reference in its entirety).

Pharmaceutical acceptable carriers are well known to those skilled in the art and include, for example, sterile saline, lactose, sucrose, calcium phosphate, gelatin, dextrin, agar, pectin, peanut oil, olive oil, sesame oil and water. Furthermore the pharmaceutical composition according to the invention may comprise one or more stabilizers such as, for example, carbohydrates including sorbitol, mannitol, starch, sucrose, dextrin and glucose, proteins such as albumin or casein, and buffers like alkaline phosphates.

It is also understood by those of skill in the art that the human PTEC cell line as disclosed herein may be used in various methods to screen a variety of test molecules/agents (also referred to as inhibitors, activators, modulators and regulators) for their ability to inhibit the binding of IL-2 to its receptor. Screening may also be done for molecules/agents that interfere with the downstream intracellular signalling system activated with the binding of IL-2 to the IL-2 receptor (i.e. c-FLIP and caspase-8 activities). Such identified compounds may also be used therapeutically as described herein.

For clarity, “inhibitors,” “activators,” “modulators,” and “regulators” refer to molecules that activate, inhibit, modulate, regulate and/or block an identified function. Any molecule having potential to activate, inhibit, modulate, regulate and/or block an identified function can be a “test molecule,” as described herein. For example, referring to IL-2 receptor function, such molecules may be identified using in vitro and in vivo assays of IL-2 and IL-2 receptor, respectively. Inhibitors are compounds that partially or totally block IL-2 receptor activity or IL-2 binding to the IL-2 receptor, respectively, decrease, prevent, or delay their activation, or desensitize its cellular response. This may be accomplished by binding to the IL-2 receptor directly or via other intermediate molecules. An antagonist as described herein such as an antibody that blocks IL-2 receptor activity, including inhibition of cellular apoptosis is considered to be such an inhibitor. Activators are compounds that bind to IL-2 receptor directly or via other intermediate molecules, thereby increasing or enhancing its activity, stimulating or accelerating its activation, or sensitizing its cellular response. An agonist of IL-2 receptor is considered to be such an activator. A modulator can be an inhibitor or activator. A modulator may or may not bind to IL-2 receptor directly; it affects or changes the activity or activation of IL-2 receptor or the sensitivity to IL-2, respectively. A modulator also may be a compound, for example, a small molecule, that inhibits transcription of IL-2 receptor mRNA. A regulator of the IL-2 receptor gene includes any element, for example, nucleic acid, peptide, polypeptide, protein, peptide nucleic acid or the like, that influence and/or control the transcription/expression of the IL-2 receptor gene, respectively, or its coding region.

### IL-2 Antagonist Treatment

Methods provided herein comprise administering an IL-2 antagonist to a patient, thereby reducing an IL-2-induced biological response that plays a role in a particular condition, that is, in inflammation and inflammatory mediated injury in tissues/organs expressing the IL-2 receptor. In particular embodiments, methods of the invention involve contacting endogenous IL-2 with an IL-2 antagonist, e.g., in an ex vivo procedure. Treatment encompasses alleviation of at least one symptom of a disorder, or reduction of disease severity of inflammation, and the like. An antagonist need not effect a complete “cure”, or eradicate every symptom or manifestation of a disease, to constitute a viable therapeutic agent. As is recognized in the pertinent field, drugs employed as therapeutic agents may reduce the severity of a given disease state, but need not abolish every manifestation of the disease to be regarded as useful therapeutic agents. One embodiment of the invention is directed to a method comprising administering to a patient an IL-2 antagonist in an amount and for a time sufficient to induce a sustained improvement over baseline of an indicator that reflects the severity of the particular inflammatory disorder. Antibodies that inhibit the binding of IL-2 to cells are discussed herein. A method for suppressing IL-2-induced activities in humans comprises administering an effective amount of such an antibody. Conditions induced by IL-2 thus may be treated. In aspects, the cells targeted are IL-2 receptor expressing cells, non-T-cells, such as epithelial cells.

As is understood in the pertinent field, antagonists are administered to a patient in a manner appropriate to the indication. Antagonists may be administered by any suitable technique, including but not limited to parenterally, topically, or by inhalation. If injected, the antagonist can be administered, for example, via intra-articular, intravenous, intramuscular, intralesional, intraperitoneal or subcutaneous routes, by bolus injection, or continuous infusion. Localized administration, e.g. at a site of disease or injury is contemplated, as are transdermal delivery and sustained release from implants. Delivery by inhalation includes, for example, nasal or oral inhalation, use of a nebulizer, inhalation of the antagonist in aerosol form, and the like. Other alternatives include eyedrops; oral preparations including pills, syrups, lozenges or chewing gum; and topical preparations such as lotions, gels, sprays, and ointments.

Use of IL-2 antagonists in ex vivo procedures is contemplated. For example, a patient's blood (bodily fluid containing IL-2) may be contacted with an antagonist that binds IL-2 ex vivo, thereby reducing the amount of IL-2 in the fluid when returned to the patient. The antagonist may be bound to a suitable insoluble matrix or solid support material. Advantageously, antagonists are administered in the form of a composition comprising at least one IL-2 antagonist and one or more additional components such as a physiologically acceptable carrier, excipient or diluent. The present invention provides such compositions comprising an effective amount of an IL-2 antagonist, for use in the methods provided herein. The compositions contain antagonist(s) in any of the forms described herein. The antagonist may be a whole antibody or an antigen-binding fragment or engineered derivative thereof, for example. For compositions containing an IL-2 receptor, the receptor may be any of the fragments, variants, or oligomers of the IL-2 receptor protein.

Compositions may, for example, comprise an antagonist together with a buffer, antioxidant such as ascorbic acid, low molecular weight polypeptide (such as those having fewer than 10 amino acids), protein, amino acid, carbohydrate such as glucose, sucrose or dextrins, chelating agents such as EDTA, glutathione, and other stabilizers and excipients. Neutral buffered saline or saline mixed with non-specific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16.sup.th Ed., Mack Publishing Company, Easton, Pa., 1980.

Kits for use by medical practitioners include an IL-2 antagonist and a label or other instructions for use in treating any of the conditions discussed herein. The kit preferably includes a sterile preparation of one or more IL-2 antagonists, which may be in the form of a composition as disclosed above, and may be in one or more vials. Dosages and the frequency of administration may vary according to such factors as the route of administration, the particular antagonist employed, the nature and severity of the disease to be treated, whether the condition is acute or chronic, and the size and general condition of the patient. Appropriate dosages can be determined by procedures known in the pertinent art, e.g. in clinical trials that may involve dose escalation studies.

An IL-2 antagonist may be administered once, or repeatedly. In particular embodiments, the antagonist is administered over a period of at least a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the antagonist is administered until the patient manifests a medically relevant degree of improvement over baseline for the chosen indicator or indicators. In embodiments of the invention involving tissues/organs for transplantation, the IL-2 antagonist may be used as a bathing solution.

Particular embodiments of the present invention involve administering an antagonist at a dosage of from about 1 ng/kg/day to about 10 mg/kg/day, more preferably from about 500 ng/kg/day to about 5 mg/kg/day, and most preferably from about 5 ug/kg/day to about 2 mg/kg/day, to a patient. In additional embodiments, an antagonist is administered to adults one time per week, two times per week, or three or more times per week, to treat the medical disorders disclosed herein. If injected, the effective amount of antagonist per adult dose may range from 1-20 mg/m², and preferably is about 5-12 mg/m². Alternatively, a flat dose may be administered; the amount may range from 5-100 mg/dose. One range for a flat dose is about 20-30 mg per dose. In one embodiment of the invention, a flat dose of 25 mg/dose is repeatedly administered by injection. If a route of administration other than injection is used, the dose is appropriately adjusted in accordance with standard medical practices. One example of a therapeutic regimen involves injecting a dose of about 20-30 mg of IL-2 antagonist one to three times per week over a period of at least three weeks, though treatment for longer periods may be necessary to induce the desired degree of improvement. For pediatric patients (age 4-17), one suitable regimen involves the subcutaneous injection of 0.4 mg/kg, up to a maximum dose of 25 mg of IL-4R, administered two or three times per week.

An antagonist is administered to the patient in an amount and for a time sufficient to induce an improvement, preferably a sustained improvement, in at least one indicator that reflects the severity of the disorder that is being treated. Various indicators that reflect the extent of the patient's illness may be assessed for determining whether the amount and time of the treatment is sufficient. Such indicators include, for example, clinically recognized indicators of disease severity, symptoms, or manifestations of the disorder in question. In most instances, an improvement is considered to be sustained if the patient exhibits the improvement on at least two occasions separated by two to four weeks. The degree of improvement generally is determined by the patient's physician, who may make this determination based on signs or symptoms, and who may also employ questionnaires that are administered to the patient, such as quality-of-life questionnaires developed for a given disease.

A patient's levels of IL-2 (and, optionally, of other TH2-type cytokines) may be monitored during and/or after treatment with an IL-2 antagonist, to detect reduction in the levels of the cytokines. For some disorders, the incidence of elevated IL-2 levels may vary according to such factors as the stage of the disease or the particular form of the disease. Known techniques may be employed for measuring IL-2 levels, e.g., in a patient's serum. Cytokine levels in blood samples may be measured by ELISA, for example.

In embodiments of methods and compositions of the invention the use of two or more different IL-2 antagonists is contemplated. In further embodiments, IL-2 antagonist(s) are administered alone or in combination with other agents useful for treating the condition with which the patient is afflicted. Examples of such agents include both proteinaceous and non-proteinaceous drugs. When multiple therapeutics are co-administered, dosages may be adjusted accordingly, as is recognized in the pertinent art. “Co-administration” and combination therapy are not limited to simultaneous administration, but include treatment regimens in which an IL-2 antagonist is administered at least once during a course of treatment that involves administering at least one other therapeutic agent to the patient. Examples of other agents that may be co-administered with IL-4 antagonists are other antibodies, cytokines, or cytokine receptors, which are chosen according to the particular inflammatory condition to be treated. Non limiting examples may be TNF antagonists and IL-17 antagonists.

### SiRNA Therapy

According to another embodiment of the invention, a siRNA targeted to inhibit expression of the IL-2 receptor in cells of tissues and organs may be used in the methods of the invention. In this aspect, is a therapeutically effective amount of a composition comprising an siRNA targeted to inhibit expression of the IL-2 receptor endogenous target gene in a cell (non-T cell) wherein the siRNA suppresses IL-2 receptor activity. One of skill in the art may construct a suitable siRNA to the IL-2 receptor as is taught in the art (Lamberton J. and Christian A. 2003. Mol. Biotechnol. June; 24(2):111-20, the entirety of the disclosure is incorporated herein by reference). Furthermore as a reference only, the mouse and human nucleotide sequence for IL-2 receptor is provided in Table 1 (Shimuzu et al., Feb. 14, 1985, Vol 13, No. 5, Nucleic Acid Research).

Briefly, RNA interference is a mechanism of post-transcriptional gene silencing. Specific gene silencing is mediated by short strands of duplex RNA of approximately 21 nucleotides in length (termed small interfering RNA or siRNA) that target the cognate mRNA sequence for degradation. While other techniques may be used to block specific molecules in vitro and in vivo, such as anti-sense oligonucleotides (Gerwitz, A. M. 1999. Curr Opin Mol Ther 1:297) and monoclonal antibodies (Drewe, E., et al., 2002. J Clin Pathol 55:81), RNAi may be used in the present invention because it provides several distinct advantages. First, mRNA degradation by siRNA is extremely efficient as only a few copies of dsRNA are necessary to activate the RNA induced silencing complex (RISC) (Martinez, J. A. et al., 2002. Cell 10:563). Once RISC is activated it can conduct multiple rounds of gene-specific mRNA cleavage. Second, RNAi is specific, in that only sequences with identity to one of the strands of dsRNA will be cleaved (Hannon, G. J. 2002. Nature 418:244). Third, the RNAi effect is long lasting and can be spread to progeny cells after replication, although a dilution effect is evident in mammalian cells (Fire, A., et al., 1998. Nature 391:806). This technique is relatively simple, giving rise to an in vitro knock down phenotype within days that can be confirmed with many antibody based detection systems (such as ELISA or Western Blotting), or if an antibody is not available, by RT-PCR or functional assays. Tissues and organs may be transformed with siRNA alone or siRNA contained within a plasmid or vector that results in the production of the siRNA against the target IL-2 receptor.

In an embodiment of the invention, the siRNA composition of the invention targeting IL-2 receptor expression is administered to the desired organ or tissue or cells by perfusion with and/or by bathing the ex vivo tissue, organ or cells in a suitable physiological solution containing the siRNA (Hamar, P., et al., Proc Natl Acad Sci 2004; 101:41). For example, a commercially available organ storage solution such as but not limited to Collins Solution, (UW)-solution, Histidine-Typtophan-Ketoglutarate (HTK) Solution, ViaSpan™ (intracellular) and Celsior solution (extracellular) may be used (Muhlbacher et al., 1999, Transplant Proc 31(5):2069-2070). Furthermore, other known additives may also be used in combination with the siRNA of the invention in the composition. For example such additives may include but not be limited to superoxide dismutase and other free radical scavengers (Baker et al,. 1999, J Surg Res 86(1):145-149; McAnulty and Huang 1996, Cryobiology 33(2): 217-225; McLaren and Friend 2003, Transpl Int 16(10):701-708), lazaroids, anti-apoptosis agents (El-Gibaly et al., 2004, Hepatology 39(6):1553-1562; Natori et al., 2003, Liver Transpl 9(3):278-284), calcium channel blockers (Arnault et al., 2003, Transplantation 76(1):77-83), intercellular adhesion molecule-1 inhibitors (Stepkowski et al., 1998, Transplantation 66(6):699-707; Chen et al., 1999, Transplantation 68(6):880-887), pentoxifylline (Randsbaek et al., 2000, Scand Cardiovasc J 34(2):201-208) and combinations thereof.

The siRNA may be used in a variety of strategies to silence the IL-2 receptor gene or combination of gene(s). For example, the following four strategies may be used: 1) Using a commercially pre-synthesized “siRNA pool” (Dharmacon Inc) consisting of 21 base-pair oligonucleotides that simultaneously target sites of the IL-2 receptor target gene; 2) Using siRNA expression vectors (psilencer™, Ambion Inc) with a pol III promoter that drives hairpin RNA expression to form a double-stranded RNA that serves as an endogenously expressed siRNA; 3) Using siRNA-expression cassettes (SEC), which are generated as PCR products consisting of a hairpin siRNA template flanked by promoter and terminator sequences. Once the SEC is transfected into cells, the hairpin siRNA is expressed from the PCR product and leads to gene silencing; 4) Use SEC-vectors. Other strategies are encompassed by the present invention as is well understood by those of skill in the art.

Depending on the particular target and the dose of siRNA delivered, partial or complete loss of function for the target IL-2 receptor gene may be achieved. A reduction or loss of gene expression in at least 50%, 60%, 70%, 80%, 90%, 95% or 99% or more of targeted cells is exemplary. Inhibition of gene expression refers to the absence (or observable decrease) in the level of protein and/or mRNA product from a target gene. Specificity refers to the ability to inhibit the target gene without manifest effects on other genes of the cell. The consequences of inhibition can be confirmed by examination of the outward properties of the cell or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS).

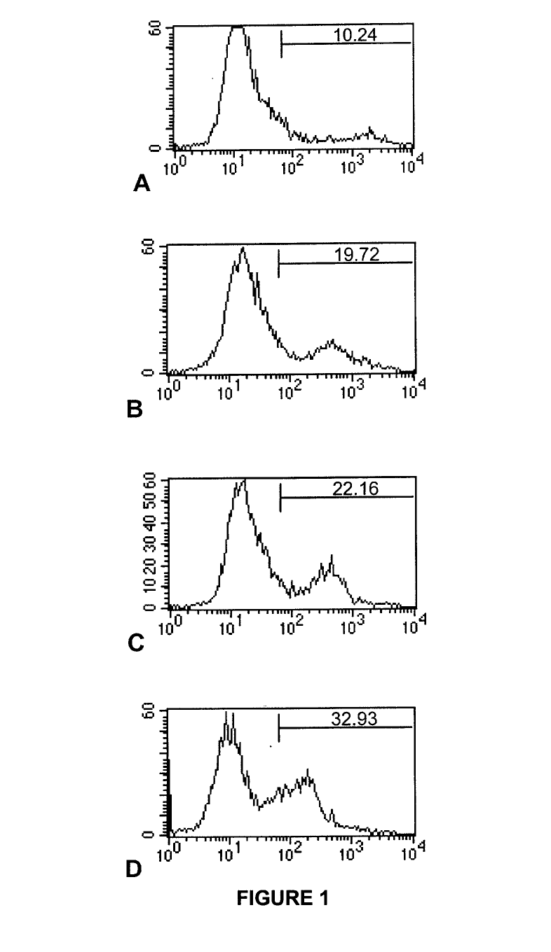

The siRNA may be administered to the tissue or organ or cells in various forms, for example (1) as a naked siRNA oligonucleotide; (2) incorporated into an siRNA expression vector which drives hairpin RNA expression to form a double stranded RNA that serves as an endogenously expressed siRNA. For example, siRNA expression vectors may be constructed with pSilencer 2.0-U6 (Ambion Inc. Austin Tex.). The specific siRNA insert oligonucleotides should be designed according to user's instruction. The oligonucliotide contains 19-mer hairpin sequences specific to the mRNA target, a loop sequence separating the two complementary domains, two 3′-end overhang necleiotide and a poly thymidine tract to terminate transcription and 5′single-stranded overhang for ligation into pSilencer with BamH1 and Hind III. Both sense and anti-sense hairpin siRNA-encoding oligonulciotides were annealed as an insert as described in Shi, Y., (2003), Trends Genet., v. 19, pp. 9-12; (3) as an siRNA expression cassette (SEC), generated as a PCR product consisting of a hairpin siRNA template flanked by promoter and terminator sequences, as described in Castanotto et al., (2002), Rna, v. 8, pp. 1454-60. Briefly, SECs were generated using a Silencer Express Kit (Ambion Inc, Austin Tex.). Sense and anti-sense hairpin siRNA template oligonucleotides for the precursor SEC were designed according to user's instruction. The oligonucleiotides contain 19-mer hairpin sequences specific to the mRNA target, a loop separating the two complementary domains, two 3′-end overhang nucleiotide. Briefly, two PCR reactions were performed to generate the precursor SEC using a Promoter Element (mouse U6) as template, a promoter PCR primer, and gene specific sense and anti-sense oligonucleotides. The first PCR product was used as template for the second PCR. The third PCR was performed to modify nucleiotides at their 5′ ends and encode EcoR I and Hind III restriction sites (FIG. 1). Taq polymerase was used in PCRs (Invetregene Inc.); and (4) an SEC incorporated into a vector. Once effective SEC has been identified, the SEC was cloned into pVP22 with Mun I (compatible with EcoR I) and Hind III sites as described in Paul (2003), Mol. Ther., v. 7, pp. 237-247.

Any of the above forms of siRNA as described herein can be used and administered for mammalian use, including animals and humans. As for the amount of siRNA for use in the composition, about 1-50 micrograms per injection can be used in animals such as mice, which is sufficient to silence genes in vivo. About up to 0.2 to 100 μg/ml siRNA, per different siRNA, in solution may be used for flushing and storing heart and kidney organs. This includes any range therein between. A dosage regime may be used as is understood by one of skill in the art. The dosage regime can be done over a period of minutes, hours or days and can use various dosages of siRNA. Of course, the amounts of siRNA used in the composition may vary depending on the particular type of tissue or organ and the size thereof and can be readily determined by one of skill in the art. Therefore, the ranges provided herein are a guide and may in fact be greater.

The siRNA may be directly introduced into the cell (i.e., intracellularly), tissue, organ, allograft or organism; or introduced extracellularly into a cavity, interstitial space, into the circulation of an organism, introduced orally, or may be introduced by bathing a cell, tissue, organ, allograft or organism in a solution containing the siRNA. The bile or biliary system, vascular or extravascular circulation, the blood or lymph system, and the cerebrospinal fluid are sites where the siRNA may be introduced. In certain embodiments of the invention, the siRNA is provided to a transplanted tissue (e.g. an organ) by perfusion. Such organs may be selected from but not limited to heart, kidneys, liver, lungs, pancreas, small intestine, skin, bone, viens and tendons. In particular embodiments, the organ is a kidney.

In further embodiments of the invention an antisense approach may be taken to downregulate IL-2 receptor expression and in this manner decrease IL-2 binding leading to inflammation. Antisense RNA is briefly defined as follows: In eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA”. Antisense RNA molecules can inhibit mRNA expression (for example, Rylova et al., Cancer Res, 62(3):801-8, 2002; Shim et al., Int. J. Cancer, 94(1):6-15, 2001).

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

TABLE 1
Human IL-2 Receptor cDNA

| | |
|---|---|
| 1 | gagagactgg atggacccac aagggtgaca gcccaggcgg accgatcttc ccatcccaca |
| 61 | tcctccggcg cgatgccaaa aagaggctga cggcaactgg gccttctgca gagaaagacc |
| 121 | tccgcttcac tgccccggct ggtcccaagg gtcaggaaga tggattcata cctgctgatg |
| 181 | tggggactgc tcacgttcat catggtgcct ggctgccagg cagagctctg tgacgatgac |
| 241 | cagccagaga tcccacacgc cacattcaaa gccatggcct acaaggaagg aaccatgttg |
| 301 | aactgtgaat gcaagagagg tttccgcaga ataaaaagcg ggtcactcta tatgctctgt |
| 361 | acaggaaact ctagccactc gtcctgggac aaccaatgtc aatgcacaag ctctgccact |
| 421 | cggaacacaa cgaaacaagt gacacctcaa cctgaagaac agaaagaaag gaaaaccaca |
| 481 | gaaatgcaaa gcccaatgca gccagtggac caagcgagcc ttccaggtga agagaagcct |
| 541 | caggcaagcc ccgaaggccg tcctgagagt gagacttcct gcctcgtcac aacaacagat |
| 601 | tttcaaatac agacagaaat ggctgcaacc atggagacgt ccatatttac aacagagyac |
| 661 | caggtagcag tggccggctg tgttttcctg ctgatcagcg tcctcctcct gagtgggctc |
| 721 | acctggcagc ggagacagag gaagagtaga agaacaatct agaaaaccaa aagaacaaga |
| 781 | atttcttggt aagaagccgg gaacagacaa cagaagtcat gaagcccaag tgaaatcaaa |
| 841 | ggtgctaaat ggtcgcccag gagacatccg ttgtgcttgc ctgcgttttg gaagctctga |
| 901 | agtcacatca caggacacgg ggcagtggca accttgtctc tatgccagct cagtcccatc |
| 961 | agagagcgag cgctacccac ttctaaatag caatttcgcc gttgaagagg aagggcaaaa |
| 1021 | ccactagaac tctccatctt attttcatgt atatgtgttc attaaagcat gaatggtatg |
| 1081 | gaactctctc caccctatat gtagtataaa gaaaagtagg tttacattca tctcattcca |
| 1141 | acttcccagt tcaggagtcc caaggaaagc cccagcacta acgtaaatac acaacacaca |
| 1201 | cactctaccc tatacaactg gacattgtct gcgtggttcc tttctcagcc gcttctgact |
| 1261 | gctgattctc ccgttcacgt tgcctaataa acatccttca agaactctgg gctgctaccc |
| 1321 | agaaatcatt ttacccttgg ctcaatcctc taagctaacc cccttccact gagccttcag |
| 1381 | tcttgaattt ctaaaaaaca gaggccatgg cagaataatc tttgggtaac ttcaaaacgg |
| 1441 | ggcagccaaa cccatgaggc aatgtcagga acagaaggat gaatgaggtc ccaggcagag |
| 1501 | aatcatactt agcaaagttt tacctgtgcg ttactaattg gcctctttaa gagttagttt |
| 1561 | ctt |

TABLE 2
Human IL-2 Receptor Genomic Sequence

| | |
|---|---|
| 1 | gaattccccc cccccccccc cgagagactg gatggaccca caagggtgac agcccaggcg |
| 61 | gaccgatctt cccatcccac atcctccggc gcgatgccaa aaagaggctg acggcaactg |
| 121 | ggccttctgc agagaaagac ctccgcttca ctgccccggc tggtcccaag ggtcaggaag |
| 181 | atggattcat acctgctgat qtggggactg ctcacgttca tcatggtgcc tggctgccag |
| 241 | gcagagctct gtgacgatga cccgccagag atcccacacg ccacattcaa agccatggcc |
| 301 | tacaaggaag gaaccatgtt gaactgtgaa tgcaagagag gtttccgcag aataaaaagc |
| 361 | gggtcactct atatgctctg tacaggaaac tctagccact cgtcctggga caaccaatgt |
| 421 | caatgcacaa gctctgccac tcggaacaca acgaaacaag tgacacctca acctgaagaa |
| 481 | cagaaagaaa ggaaaaccac agaaatgcaa agtccaatgc agccagtgga ccaagcgagc |
| 541 | cttccaggtc actgcaggga acctccacca tgggaaaatg aagccacaga gagaatttat |
| 601 | catttcgtgg tggggcagat ggtttattat cagtgcgtcc agggatacag ggctctacac |
| 661 | agaggtcctg ctgagagcgt ctgcaaaatg acccacggga agacaaggtg gacccagccc |
| 721 | cagctcatat gcacaggtga aatggagacc agtcagtttc caggtgaaga gaagcctcag |
| 781 | gcaagccccg aaggccgtcc tgagagtgag acttcctgcc tcgtcacaac aacagatttt |
| 841 | caaatacaga cagaaatggc tgcaaccatg gagacgtcca tatttacaac agagtaccag |
| 901 | gcagcagtgg ccggctgtgt tttcctgctg atcagcgtcc tcctcctgag tgggctcacc |
| 961 | tggcagcgga gacagaggaa gagtagaaga acaatctaga aaaccaaaag aacaagaatt |
| 1021 | tcttggtaag aagccgggaa cagacaacag aagtcatgaa gcccaagtga aatcaaaggt |
| 1081 | gctaaatggt cgcccaggag acatccgttg tgcttgcctg cgttttggaa gctctgaagt |
| 1141 | cacatcacag gacacggggc agtggcaacc ttgtctctat gccagctcag tcccatcaga |
| 1201 | gagcgagcgc tacccacttc taaatagcaa tttcgccgtt gaagaggaag ggcaaaacca |
| 1261 | ctagaactct ccatcttatt ttcatgtata tgtgttcatt aaagcatgaa tggtatggaa |
| 1321 | ctctctccac cctatatgta gtataaagaa aagtaggttt acattcatct cattccaact |
| 1381 | tcccagttca ggagtcccaa ggaaagcccc agcactaacg taaatacaca acacacacac |
| 1441 | tctaccctat acaactggac attgtctgcg tggttccttt ctcagccgct tctgactgct |
| 1501 | gattctcccg ttcacgttgc ctaataaaca tccttcaaga actctgggct gatacccaga |
| 1561 | aatcatttta cccttggctc aatcctctaa gctaaccccc ttctactgag ccttcagtct |
| 1621 | tgaatttcta aaaaacagag gccatggcag aataatcttt gggtaacttc aaaacggggc |
| 1681 | agccaaaccc atgaggcaat gtcaggaaca gaaggatgaa tgaggtccca ggcagagaac |
| 1741 | catacttagc aaagttttac ctgcgcgtta ctaattggcc tctttaagag ttagtttctt |
| 1801 | tgggattgct atgaatgata ccctgaattt ggcctgcact aatttgatgt ttacaggtgg |
| 1861 | acacacaagg tgcaaatcaa tgcgtacgtt tcctgagaag tgtctaaaaa caccaaaaag |
| 1921 | ggatccgtac attcaatgtt tatgcaagga aggaaagaaa gaaggaagtg aagagggaga |
| 1981 | agggatggag gtcacactgg tagaacgtaa ccacggaaaa gagcgcatca ggcctggcac |
| 2041 | ggtggctcag gcctataacc ccagctccct aggagaccaa ggcgggagca tctcttgagg |
| 2101 | ccaggagttt gagaccagcc tgggcagcat agcaagacac atccctacaa aaaattagaa |
| 2161 | attggctgga tgtggtggca tacgcctgta gtcctagcca ctcaggaggc tgaggcagga |
| 2221 | ggattgcttg agcccaggag ttcgaggctg cagtcagtca tgatggcacc actgcactcc |
| 2281 | agcctgggca acagagcaag atcctgtctt taaggaaaaa aagacaaggg aattc |

### Examples

### Inhibition of the IL-2 Receptor

Previously cloned and immortalized PTEC (NG) via origin defective SV40 transfection of B6 male mouse primary PTEC were prepared, and this cell line was used for all in vitro mouse experiments (Du et al., Kidney International, Vol. 67 (2005), pp. 1-13). Human transformed PTEC cell line HK-2 (ATCC, Manassas, Va., USA) were used for human in vitro experiments.

NG cells were cultured in K1 media with 5% FBS of confluence and plated to 24 well flat bottom plates at a density of 2×10⁵ cells/mL overnight. Plates were washed twice and mouse recombinant IL-2 in K1 serum free media was overlayed. In selected wells, pre-treatment with mouse anti-CD25 MAb (Cedarlane, Hornby, Ontario, CAN) or basiliximab (Simulect, Novartis, Dorval, Quebec, CAN) for 1 hour was performed. The plates were incubated at 37° C. for 24 hours, followed by analysis with Annexin V-FITC/7AAD FACS.

HK-2 cells were cultured in keratinocyte serum free media (GIBCO, Carlsbad, Calif., USA) with 5 ng/mL bovine pituitary extract to 80% confluence, washed, trypsinized and plated on 24-well flat bottom collagen coated culture plates at a density of 2×10⁵/mL overnight. Plates were then washed twice and human recombinant IL-2 (R&D Systems, Minneapolis, Minn., USA) in serum free media was overlayed at increasing concentrations (FIG. 1). The plates were then incubated at 37° C. for 24 hours, following which Annexin V-FITC/7 AAD FACS analysis was performed for dose response.

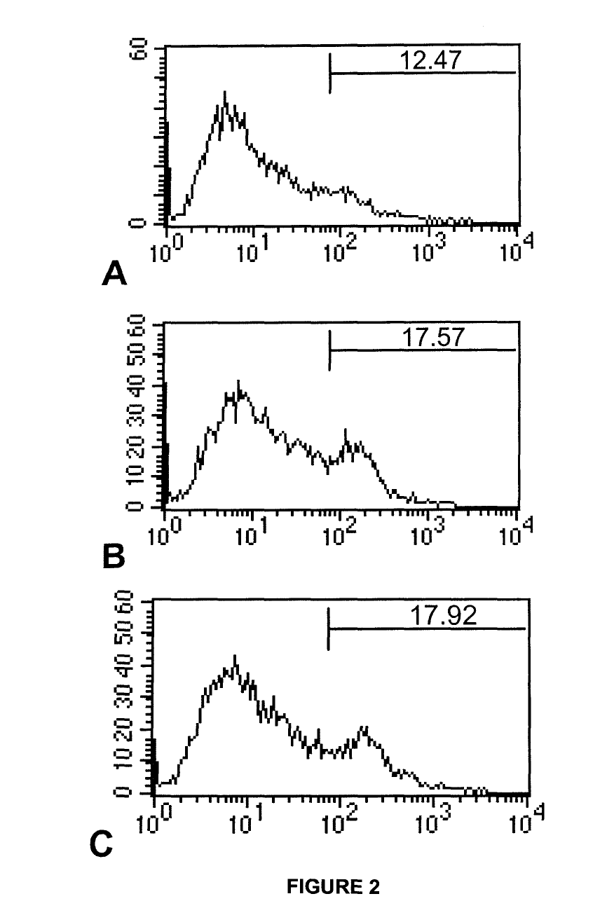

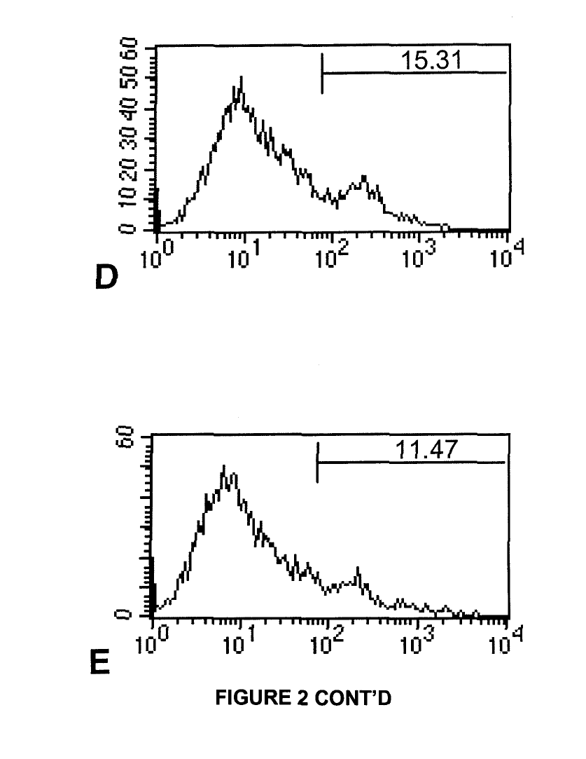

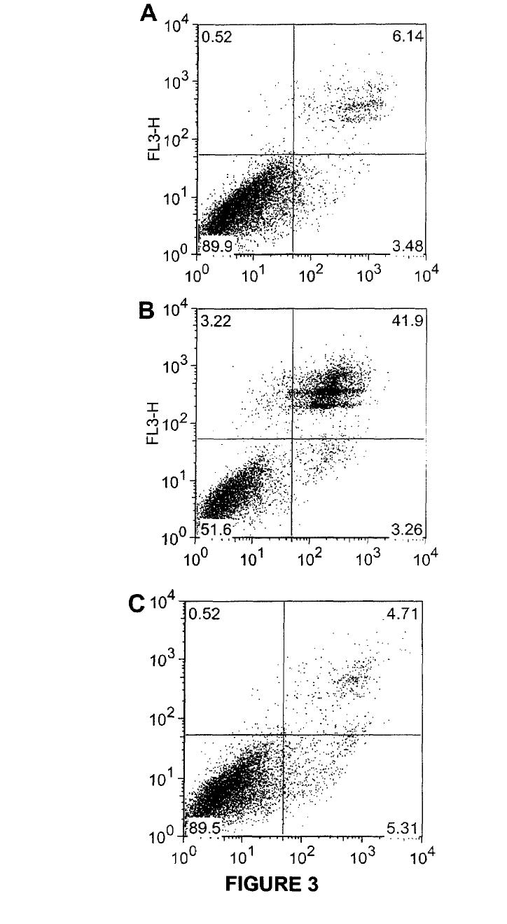

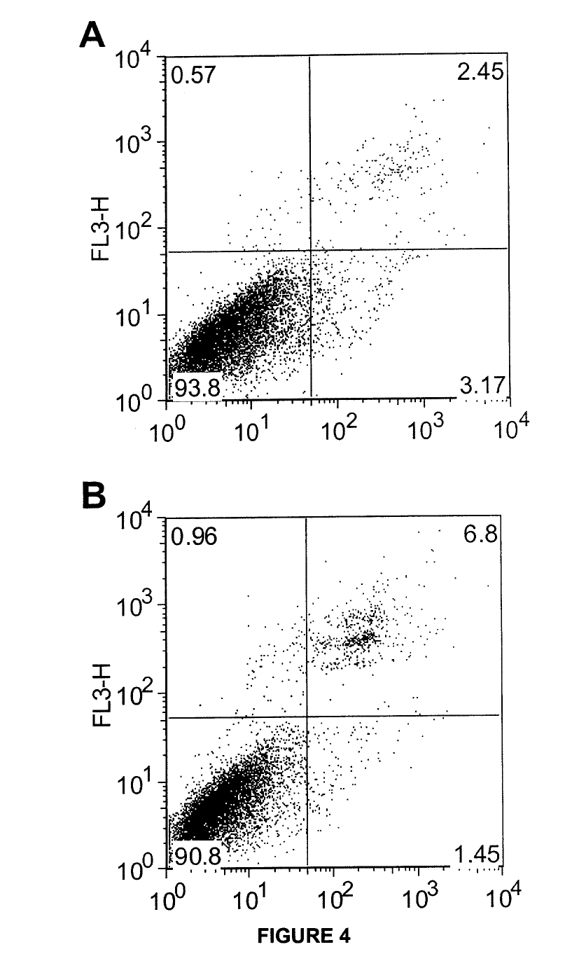

Blocking studies (FIGS. 2-4) were carried in similar fashion, plating 2×10⁵ HK-2 cells/mL in 24 well culture plates overnight, followed by pre-treatment of monolayers with 10 μg/mL basiliximab for 1 hour. Wells were washed and 20 ng/mL of human recombinant IL-2 was overlayed and the plates incubated for 24 hours at 37° C. Apoptosis was assayed through Annexin V-FITC/7 AAD FACS analysis.

The IL-2 binding assay may be performed as already described herein or as described in MOREAU et al. (1995b) Mol. Immunol. 32:1047-1056 for various muteins. ¹²⁵I-labelled IL-2 binding to different cell lines is studied. Inhibition experiments were performed at concentration of ¹²⁵I-labelled IL-2 giving between 50 to 70% maximum binding. The effects of different muteins can be analyzed after 1 hr preincubation at 4°. followed by incubation with ¹²⁵I-labelled IL-2 (3 hr at 4° C.). In experiments nonspecific binding is determined. The data is expressed as % inhibitory capacity of the different mutein versus wild type protein.

### IL-2 Receptor Blockade—Organ Transplantation

The IL-2 receptor is blocked as described herein to prevent the engagement of IL-2 to epithelial cells, notably in the kidney but elsewhere as well, to attenuate or prevent IL-2 mediated injury. Kidney epithelial cells respond to IL-2 by undergoing apoptosis. The effect is both direct through the activation of caspase-8 enzyme activation within the cell which leads to death or by inhibiting endogenous proteins that augment survival during inflammation. The result of excessive kidney cell death is organ dysfunction, which can be measured clinically.

IL-2 blocking agents, such as the antagonists described herein which includes anti-receptor antibody and siRNA to prevent receptor expression, are administered to organs awaiting transplantation by ex vivo administration in the organ perfusion solution. The dosage of antibody is determined by the amount of perfusion solution, to achieve saturation of the receptor as is understood and readily determinable. The range of antibody for delivered to cell systems in vitro for blocking studies range from about 0.1 to about 10 μg/ml. This level is readily achievable using current antibody preparations. The level of siRNA is similarly determined from current experimental data in other RNA silencing systems. The use of siRNA may require rapid infusion into the perfused organ to exert an effect, similar to the use of siRNA in intact animals. Alternatively siRNA is administered as part of a targeting complex in the form of a plasmid, or bound to lipids to reduce the dose required or to more efficiently transfer siRNA to cells within the organ. The introduction of siRNA or antibody in perfused organs targets IL-2 receptors on all cells including epithelial cells.

Alternatively, IL-2 blocking agents such as antibody are also administered to donors following declaration of death (post vivo), and reach target organs by circulation. This utilizes doses of antibody currently in use clinically for recipients at the time of transplant, but would not be limited by toxicity issues to allow supra physiologic doses. Currently the use of basiliximab is limited to 20 mg infusion per exposure but this may be increased to much higher doses in donors. The additional advantage is that the body temperature of 37° C. optimizes binding of IgG blocking antibody, which binds best at normal body temperatures. Similarly infusion of siRNA is not be limited by systemic toxicity in donors and the efficiency of transfer to cells may be increased at 37° C.

Alternatively the IL-2 receptor blocking agents are delivered to recipients post transplant, in doses that have been approved for clinical use. Basiliximab (Simulect®, Novartis, Basle, Switzerland) is a chimeric (human/mouse) mAb (molecular mass approximately 144 kDa) of the IgG1κ isotype. The purified preparation is formulated as a lyophilisate that has undergone heat treatment to inactivate viruses, and meets all quality-controlled criteria for mAbs intended for use in humans. When reconstituted with 5 ml of saline, the ampule contains 20 mg of basiliximab which is further diluted to 50 ml for infusion for about 20-30 minutes. Administration of the first dose is 20 mg usually immediately before transplant surgery or on the first day and a second dose of about 20 mg is typically administered on day 4 after transplantation. [Kahan, Barry D. Rajagopalan, P. R. Hall, Michael; United States Simulect Renal Study Group, REDUCTION OF THE OCCURRENCE OF ACUTE CELLULAR REJECTION AMONG RENAL ALLOGRAFT RECIPIENTS TREATED WITH BASILIXIMAB, A CHIMERIC ANTI-INTERLEUKIN-2-RECEPTOR MONOCLONAL ANTIBODY Transplantation olume 67(2), 27 Jan. 1999, pp 276-284]. The duration of therapy is limited to the immediate post transplant period as this is the greatest risk for IL-2 injury related to ischemia-reperfusion of the graft.

The 20 mg dose was determined by the level of saturation of IL-2 receptor on T lymphocytes, however, it is expected that similar levels of blocking antibody are effective on kidney epithelial cells which express lower levels of receptor. However the dosage could be increased without any anticipated toxicity in the immediate post operative period. Similarly IL-2 receptor antibody could be administered to patients with inflammation in which IL-2 may be having a deleterious effect on IL-2R bearing epithelial cells. This would include all organs as all organs contain epithelial cells which are likely to respond to IL-2. Examples include bowel cells during inflammatory bowel disease, thyroid during thyroiditis and lung during pulmonary inflammation.

Alternatively IL-2 receptor blockade is used in transplantation during acute immune rejection of solid organ grafts, to reduce or prevent organ injury while rejection is being controlled by other agents such as steroids or anti T cell antibodies. The blocking agents are delivered to recipients with rejection in the same doses that have been approved for clinical use for prophylaxis against acute rejection.

### Determination of IL-2 Blockade in Organ Transplantation

The effect of the IL-2 receptor blocking therapy is measured by a protective impact on renal function when compared to non treated controls such as for example measuring the production of urine post-transplant. [Kahan, Barry D. Rajagopalan, P. R. Hall, Michael; United States Simulect Renal Study Group, REDUCTION OF THE OCCURRENCE OF ACUTE CELLULAR REJECTION AMONG RENAL ALLOGRAFT RECIPIENTS TREATED WITH BASILIXIMAB, A CHIMERIC ANTI-INTERLEUKIN-2-RECEPTOR MONOCLONAL ANTIBODY Transplantation volume 67(2), 27 Jan. 1999, pp 276-284]. The incidences of delayed graft function is also defined by the need for hemodialysis within the first 7 postoperative days. Acute rejection typically appears after 7 days in first transplant patients. Furthermore, serum creatine values are measured to assess renal function. In the immediate post transplant period, improved kidney function is measured by conventional clinical parameters (urine output, serum creatinine, glomerular filtration rate—GFR) and as well by biomarkers such as NGAL excretion and renal tubular cell enzymuria, as markers of epithelial cell injury or death. In treatment of rejection episodes, these are measured serially to determine the return of function to baseline values, prior to rejection. It is well accepted that failure to return to baseline values post kidney transplant rejection, represents the greatest risk to patients developing long term transplant dysfunction, while return to baseline negates any long term implications of that rejection on chronic loss.

In other organs systems in which IL-2 injury is attempted to be blocked by IL-2 receptor blocking agents, conventional clinical parameters representative of the organ function demonstrates an improvement over cases in which IL-2 receptor blocking was not used.

Although preferred embodiments of the invention have been described herein in detail, it will be understood by those skilled in the art that variations may be made thereto without departing from the spirit of the invention.

1. A method of treating an inflammatory disorder/condition comprising the step of administering to a mammal a therapeutically effective amount of a composition comprising at least one agent that inhibits IL-2 receptor activation in cells expressing the IL-2 receptor.

2. The method of claim 1, wherein said agent is an IL-2 blocker or IL-2 receptor blocker.

3. The method of claim 1, wherein said IL-2 blocker or IL-2 receptor blocker is used to block IL-2 receptor activation in non T-cells.

4. The method of claim 3, wherein said non T-cells are epithelial cells.

5. The method of claim 1, wherein said agent is selected from the group consisting of an IL-2 receptor antibody, a variant of IL-2 that acts as an antagonist to the IL-2 receptor and a siRNA targeted to inhibit expression of the IL-2 receptor on said cells.

6. The method of claim 5, wherein said agent is an IL-2 receptor antibody.

7. The method of claim 6, wherein said antibody is monoclonal or polyclonal.

8. The method of claim 1, wherein said cells present in a tissue selected from the group consisting of heart, kidney, liver, lung, pancreas, small intestine, skin, bone, vein and tendon.

9. The method of claim 8, wherein said cells are kidney tubular epithelial cells.

10. The method of claim 1, wherein said inflammatory disorder/condition is selected from the group consisting of lupus, nephritis, inflammatory bowel disease and inflammation associated with transplanted organs.

11. The method of claim 10, wherein said transplanted organ is a kidney.

12. The method of claim 1, wherein said composition further comprises one or more of a pharmaceutically acceptable carrier and may be provided intra-articularly, intravenously, intramuscularly, intralesionally, intraperitoneally, and subcutaneously.

13. The method of claim 12, wherein said composition is provided by bolus injection or continuous infusion.

14. The method of claim 1, wherein said treatment is continuous or intermittent.

15. The method of claim 14, wherein said agent is provided in a daily amount of about 0.1 μmol to about 100 μmol per kg body weight, for at least a time effective to treat the subject.

16. The method of claim 14, wherein said composition is used to bathe/store/reperfusion of an organ for transplantation.

17. The method of claim 16, wherein said agent is an IL-2 receptor antibody provided at about 20 mg infusion to said organ.

18. A method for increasing the viability of a tissue/organ allograft in a mammal, said method comprising:
blocking IL-2 receptor activity of epithelial cells of said tissue/organ allograft by providing a composition comprising at least one agent that inhibits IL-2 receptor activation in said cells.

19. The method of claim 18, wherein said tissue/organ is immersed or bathed in said composition for a time effective to inhibit IL-2 receptor activation.

20. The method of claim 18, wherein said composition is administered to said mammal prior to tissue/organ allograft and/or after said tissue/organ allograft.

21. The method of claim 18, wherein said method inhibits/minimizes apoptosis of said epithelial cells.

22. The method of claim 18, wherein said agent is selected from the group consisting of an IL-2 receptor antibody, a variant of IL-2 that acts as an antagonist to the IL-2 receptor and a siRNA targeted to inhibit expression of the IL-2 receptor on said cells.

23. The method of claim 22, wherein said agent is an IL-2 receptor antibody.

24. The method of claim 23, wherein said antibody is monoclonal or polyclonal.

25. The method of claim 18, wherein said tissue/organ is selected from the group consisting of heart, kidney, liver, lung, pancreas, small intestine, skin, bone, vein and tendon.

26. The method of claim 25, wherein said cells are kidney tubular epithelial cells.

27. The method of claim 26, wherein said method prevents kidney allograft rejection in a mammal receiving such allograft.

28. The method of claim 5 or claim 22, wherein said siRNA molecule is delivered in the form of a naked oligonucleotide, sense molecule, antisense molecule, or a vector, and wherein the siRNA interacts with the IL-2 receptor gene or its transcripts on non-T cells.

29. The method of claim 28, wherein the vector is a plasmid, cosmid, bacteriophage, or a virus.

30.-49. (canceled)


SEQUENCE LISTING (2 sequences)

SEQ ID NO: 1 - DNA, 1563 bp, Homo sapiens
gagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtgaagagaagcctcaggcaagccccgaaggccgtcctgagagtgagacttcctgcctcgtcacaacaacagattttcaaatacagacagaaatggctgcaaccatggagacgtccatatttacaacagagyaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttccactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctt

SEQ ID NO: 2 - DNA, 2335 bp, Homo sapiens
gaattccccccccccccccccgagagactggatggacccacaagggtgacagcccaggcggaccgatcttcccatcccacatcctccggcgcgatgccaaaaagaggctgacggcaactgggccttctgcagagaaagacctccgcttcactgccccggctggtcccaagggtcaggaagatggattcatacctgctgatgtggggactgctcacgttcatcatggtgcctggctgccaggcagagctctgtgacgatgacccgccagagatcccacacgccacattcaaagccatggcctacaaggaaggaaccatgttgaactgtgaatgcaagagaggtttccgcagaataaaaagcgggtcactctatatgctctgtacaggaaactctagccactcgtcctgggacaaccaatgtcaatgcacaagctctgccactcggaacacaacgaaacaagtgacacctcaacctgaagaacagaaagaaaggaaaaccacagaaatgcaaagtccaatgcagccagtggaccaagcgagccttccaggtcactgcagggaacctccaccatgggaaaatgaagccacagagagaatttatcatttcgtggtggggcagatggtttattatcagtgcgtccagggatacagggctctacacagaggtcctgctgagagcgtctgcaaaatgacccacgggaagacaaggtggacccagccccagctcatatgcacaggtgaaatggagaccagtcagtttccaggtgaagagaagcctcaggcaagccccgaaggccgtcctgagagtgagacttcctgcctcgtcacaacaacagattttcaaatacagacagaaatggctgcaaccatggagacgtccatatttacaacagagtaccaggtagcagtggccggctgtgttttcctgctgatcagcgtcctcctcctgagtgggctcacctggcagcggagacagaggaagagtagaagaacaatctagaaaaccaaaagaacaagaatttcttggtaagaagccgggaacagacaacagaagtcatgaagcccaagtgaaatcaaaggtgctaaatggtcgcccaggagacatccgttgtgcttgcctgcgttttggaagctctgaagtcacatcacaggacacggggcagtggcaaccttgtctctatgccagctcagtcccatcagagagcgagcgctacccacttctaaatagcaatttcgccgttgaagaggaagggcaaaaccactagaactctccatcttattttcatgtatatgtgttcattaaagcatgaatggtatggaactctctccaccctatatgtagtataaagaaaagtaggtttacattcatctcattccaacttcccagttcaggagtcccaaggaaagccccagcactaacgtaaatacacaacacacacactctaccctatacaactggacattgtctgcgtggttcctttctcagccgcttctgactgctgattctcccgttcacgttgcctaataaacatccttcaagaactctgggctgctacccagaaatcattttacccttggctcaatcctctaagctaacccccttctactgagccttcagtcttgaatttctaaaaaacagaggccatggcagaataatctttgggtaacttcaaaacggggcagccaaacccatgaggcaatgtcaggaacagaaggatgaatgaggtcccaggcagagaatcatacttagcaaagttttacctgtgcgttactaattggcctctttaagagttagtttctttgggattgctatgaatgataccctgaatttggcctgcactaatttgatgtttacaggtggacacacaaggtgcaaatcaatgcgtacgtttcctgagaagtgtctaaaaacaccaaaaagggatccgtacattcaatgtttatgcaaggaaggaaagaaagaaggaagtgaagagggagaagggatggaggtcacactggtagaacgtaaccacggaaaagagcgcatcaggcctggcacggtggctcaggcctataaccccagctccctaggagaccaaggcgggagcatctcttgaggccaggagtttgagaccagcctgggcagcatagcaagacacatccctacaaaaaattagaaattggctggatgtggtggcatacgcctgtagtcctagccactcaggaggctgaggcaggaggattgcttgagcccaggagttcgaggctgcagtcagtcatgatggcaccactgcactccagcctgggcaacagagcaagatcctgtctttaaggaaaaaaagacaagggaattc